(19) United States
(12) Patent Application Publication
(10) Pub. No.: US 2026/0240593 A1
(43) Pub. Date: Aug. 20, 2026

(54) LEAFLET LACERATION DEVICE

(71) Applicant: Boston Scientific Scimed, Inc., Maple Grove, MN (US)

(72) Inventors: Lauren Koon, Etobicoke (CA); Steven Kinio, Pickering (CA)

(73) Assignee: BOSTON SCIENTIFIC SCIMED, INC., MAPLE GROVE, MN (US)

(21) Appl. No.: 19/543,360

(22) Filed: Feb. 18, 2026

Related U.S. Application Data
(60) Provisional application No. 63/759,695, filed on Feb. 18, 2025.

(51) Int. Cl. A61B 18/14; A61B 17/00; A61B 17/32; A61B 18/00

(52) U.S. Cl. CPC A61B 18/1492; A61B 17/00234; A61B 17/32; A61B 2017/00243; A61B 2017/00544; A61B 2017/00557; A61B 2018/00077; A61B 2018/00178; A61B 2018/00184; A61B 2018/00285; A61B 2018/00369; A61B 2018/00595; A61B 2018/00601; A61B 2018/1407; A61B 2018/1412

(57) ABSTRACT

A medical device for lacerating valve leaflets includes an elongate shaft with a distal region and an expandable element secured to the distal region. The expandable element moves between collapsed and expanded configurations. The device has a guideway along the expandable element and a laceration member slidingly disposed within the guideway. A translation member extends relative to the elongate shaft and couples to the laceration member to enable translation relative to the guideway. The laceration member can be either an RF laceration member or a mechanical laceration member that moves along a predetermined path relative to the expandable element. The mechanical laceration member may include an atraumatic edge along its distal region and a cutting blade along its proximal region. When in the expanded configuration, the expandable element urges the cutting element into contact with the valve leaflet.

### CROSS REFERENCE TO RELATED APPLICATIONS

This application claims the benefit of priority of U.S. Provisional Application No. 63/759,695 filed Feb. 18, 2025, the entire disclosure of which is hereby incorporated by reference.

### TECHNICAL FIELD

The present disclosure relates generally to medical devices. More particularly, the present disclosure pertains to medical devices for lacerating cardiac valve leaflets.

### BACKGROUND

A wide variety of intracorporeal medical devices have been developed for medical use, and more specifically for intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for lacerating cardiac valve leaflets. An example may be found in a medical device for lacerating valve leaflets. The medical device includes an elongate shaft that extends proximally from a distal region and an expandable element that is secured to the distal region. The expandable element is moveable between a collapsed configuration and an expanded configuration. A guideway extends along the expandable element and a laceration member is slidingly disposed within the guideway. A translation member extends relative to the elongate shaft and is coupled to the laceration member such that translating the translation member causes the laceration member to be translated relative to the guideway.

Alternatively or additionally, the expandable element may include an inflatable balloon.

Alternatively or additionally, the laceration member may include an electrocautery electrode.

Alternatively or additionally, the laceration member may be adapted to lacerate a valve leaflet using RF energy.

Alternatively or additionally, the translation member may be adapted to provide an electrical connection with the electrocautery electrode.

Alternatively or additionally, the medical device may further include an electrical conductor that provides an electrical connection with the electrocautery electrode.

Alternatively or additionally, the laceration member may include a cutting blade.

Alternatively or additionally, the cutting blade may be disposed along a proximal region of the laceration member and a distal region of the laceration member may be atraumatic.

Alternatively or additionally, the laceration member may further include a puncture feature.

Alternatively or additionally, the medical device may further include a lumen that accommodates the translation member therethrough. The guideway may include a slot formed along the lumen.

Alternatively or additionally, the expandable element may urge the laceration member into contact with the valve leaflet when the expandable element is in the expanded configuration.

Alternatively or additionally, the valve leaflets may include native valve leaflets.

Another example may be found in a medical device for lacerating valve leaflets. The medical device includes an elongate shaft that extends proximally from a distal region and an expandable element that is secured to the distal region. The expandable element is moveable between a collapsed configuration and an expanded configuration. An RF laceration member is disposed relative to the expandable element. An actuation mechanism is coupled to the RF laceration member to move the RF laceration member along a predetermined path relative to the expandable element.

Alternatively or additionally, the medical device may further include a guideway defining the predetermined path.

Alternatively or additionally, the RF laceration member may include a round electrode.

Alternatively or additionally, the actuation member may include a pull wire.

Alternatively or additionally, the actuation member may include a push wire.

Another example may be found in a medical device for lacerating valve leaflets. The medical device includes an elongate shaft that extends proximally from a distal region and an expandable element that is secured to the distal region. The expandable element is moveable between a collapsed configuration and an expanded configuration. A mechanical laceration member is disposed relative to the expandable element. An actuation mechanism is coupled to the laceration member to move the mechanical laceration member along a predetermined path relative to the expandable element.

Alternatively or additionally, the medical device may further include a guideway that defines the predetermined path.

Alternatively or additionally, the mechanical laceration member includes an atraumatic edge along a distal region of the mechanical laceration member and a cutting blade along a proximal region of the mechanical laceration member.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing an aortic heart valve positioned within a native valve annulus of a heart;

FIG. 2 is a schematic view of an illustrative medical device;

FIG. 3 is a schematic view of a portion of the illustrative medical device of FIG. 2, with a laceration member shown in an initial position;

FIG. 4 is a schematic view of a portion of the illustrative medical device of FIG. 2, with the laceration member shown in a final position;

FIG. 5 is a cross-sectional view taken along the line 5-5 of FIG. 3;

FIG. 6 is a schematic view of a portion of the illustrative medical device of FIG. 2;

FIG. 7 is a schematic view of a portion of the illustrative medical device of FIG. 2;

FIG. 8 is a schematic view of a portion of the illustrative medical device of FIG. 2;

FIG. 9 is a schematic view of a portion of the illustrative medical device of FIG. 2;

FIG. 10 is a schematic view of a portion of the illustrative medical device of FIG. 2;

FIG. 11 is a schematic view of an illustrative medical device; and

FIG. 12 is a schematic view of a portion of an illustrative medical device.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove or excise one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from porcine or bovine pericardium, or may be polymeric. In some instances, artificial valve leaflets may be made of polymers such as Dacron or Gore-Tex. As discussed here, reference to a valve leaflet may refer to either a native valve leaflet or an artificial valve leaflet.

In some instances, a medical device may be adapted for lacerating valve leaflets. The medical device may include an elongate shaft that extends proximally from a distal region and an expandable element that is secured to the distal region. The expandable element is moveable between a collapsed configuration and an expanded configuration. A guideway extends along the expandable element. A laceration member is slidingly disposed within the guideway. A translation member extends relative to the elongate shaft and is coupled to the laceration member such that translating the translation member causes the laceration member to be translated relative to the guideway.

In some cases, the expandable element may include an inflatable balloon. In some cases, the laceration member may include an electrocautery electrode. The laceration member may be adapted to lacerate a valve leaflet using RF (radiofrequency) energy. In some cases, the translation member may be adapted to provide an electrical connection with the electrocautery electrode. In some cases, the medical device may further include an electrical conductor that provides an electrical connection with the electrocautery electrode. In some cases, the electrocautery electrode may be a monopolar electrode, meaning that in use, there may be a grounding pad that is placed on the patient in order to complete a circuit. In some cases, the electrocautery electrode may be a bipolar electrode. Bipolar grounding may be achieved by locating a ground electrode somewhere on the medical device. Bipolar grounding may be achieved by grounding a metallic portion of the track or guideway. As another option, the electrode itself could have a cutting electrode and a grounding electrode that are separated by an electrical insulator.

In some cases, the laceration member may include a cutting blade. The cutting blade may be disposed along a proximal region of the laceration member. A distal region of the laceration member may be atraumatic. In some cases, the laceration member may further include a puncture feature. In some cases, the medical device may further include a lumen that accommodates the translation member extending therethrough. In some cases, the guideway may include a slot formed along the lumen. In some cases, the expandable element may urge the cutting element into contact with the valve leaflet when the expandable element is in the expanded configuration. In some cases, the valve leaflets may include native valve leaflets.

In some instances, a medical device may be adapted for lacerating valve leaflets. The medical device may include an elongate shaft that extends proximally from a distal region and an expandable element that is secured to the distal region. The expandable element is moveable between a collapsed configuration and an expanded configuration. An RF laceration member is disposed relative to the expandable element. An actuation mechanism is coupled to the RF laceration member in order to move the RF laceration member along a predetermined path relative to the expandable element.

In some cases, the medical device may further include a guideway that defines the predetermined path. The RF laceration member may include a round electrode. In some cases, the actuation member may include a pull wire. In some cases, the actuation member may include a push wire.

In some instances, a medical device may be adapted for lacerating valve leaflets. The medical device may include an elongate shaft that extends proximally from a distal region and an expandable element that is secured to the distal region. The expandable element is moveable between a collapsed configuration and an expanded configuration. A mechanical laceration member is disposed relative to the expandable element. An actuation mechanism is coupled to the laceration member to move the mechanical laceration member along a predetermined path relative to the expandable element.

In some cases, the medical device may further include a guideway that defines the predetermined path. In some cases, the mechanical laceration member may include an atraumatic edge along a distal region of the mechanical laceration member. In some cases, the mechanical laceration member may include a cutting blade along a proximal region of the mechanical laceration member.

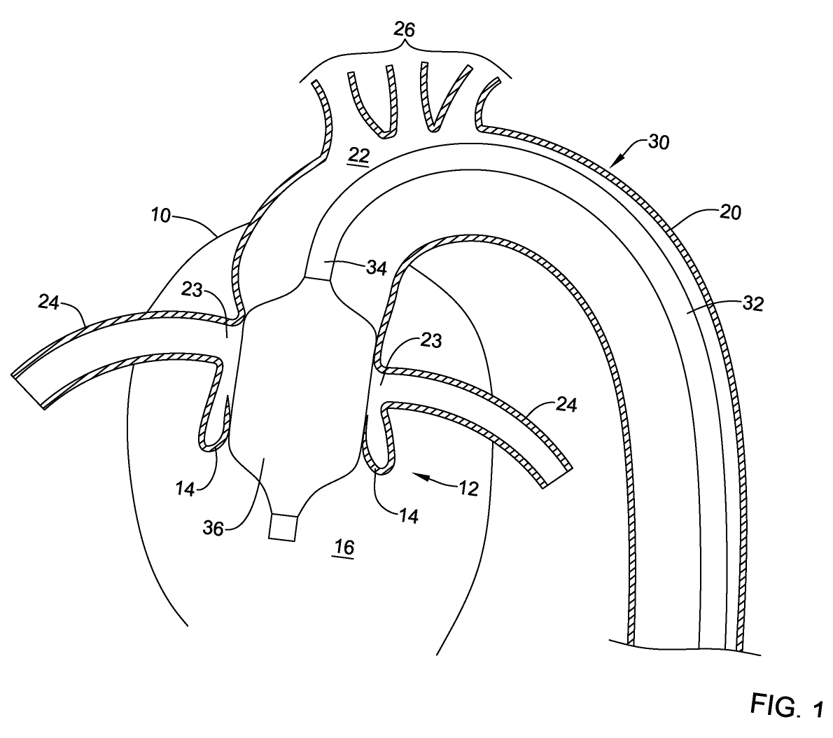

FIG. 1 is a schematic partial cut-away view of a portion of a patient’s heart 10 including an aortic valve 12 having native valve leaflets 14 disposed within and/or extending from a native valve annulus, a left ventricle 16, and certain connected vasculature, such as an aorta 20 connected to the aortic valve 12 of the patient’s heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. While the aortic valve 12 includes a total of three native valve leaflets 14, only two are visible in the illustrated cutaway view. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

As shown in FIG. 1, a medical device 30 has been advanced through the aorta 20 and through the aortic arch 22 to a position in which the medical device 30 extends through the native valve leaflets 14. The medical device 30 includes an elongate shaft 32 that extends proximally from a distal region 34. While not shown, in some cases the elongate shaft 32 may include one or more braids and/or one or more coils in order to enhance torqueability. An expandable element 36 is secured to the distal region 34. As shown, the medical device 30 is a balloon catheter, and the expandable element 36 is shown as being an inflatable balloon. In some cases, as will be discussed, the expandable element 36 may not be an inflatable balloon, and may instead include an expandable framework. Regardless, the expandable element 36 may be considered as being movable between a collapsed configuration (a deflated configuration in the case of an inflatable balloon) and an expanded configuration (an inflated configuration in the case of an inflatable balloon), as shown for example in FIG. 1. While the medical device 30 is shown compressing the native valve leaflets 14, it will be appreciated that the medical device 30 may also be used to compress artificial valve leaflets in a previously implanted replacement heart valve before implanting a new replacement heart valve within the previously implanted replacement heart valve. Some non-limiting examples of a replacement heart valves with which the medical device 30 may be utilized include the ACURATE NEO2™, the ACURATE PRIME™, and/or family members thereof from Boston Scientific of Marlborough, MA, USA.

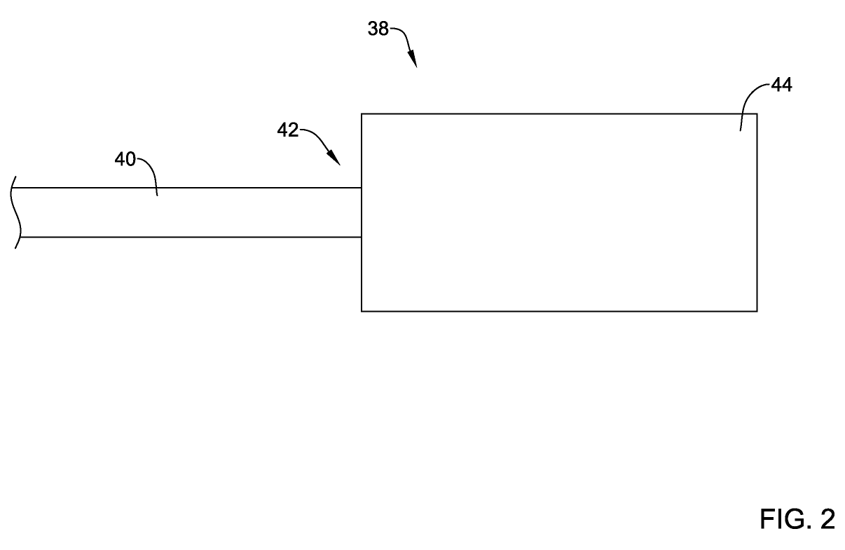

FIG. 2 is a schematic view of an illustrative medical device 38 for lacerating valve leaflets such as the native valve leaflets 14 (or artificial valve leaflets in a previously implanted replacement heart valve). The medical device 38 may be considered as a more generic example of the medical device 30 shown in FIG. 1. In some cases, there is a desire to lacerate one or more of the valve leaflets 14 in order to make it less likely that subsequent implantation of a replacement heart valve results in occluding or even partially occluding one or more of the coronary ostia 23. The medical device 38 includes an elongate shaft 40 that extends proximally from a distal region 42. An expandable element 44 is secured to the distal region 42. The expandable element 44 may be an inflatable balloon such as the inflatable balloon 36 shown in FIG. 1. In some cases, the expandable element 44 may not be an inflatable balloon, but may instead be or include an expandable framework. The medical device 38 may include a laceration member (not shown in FIG. 2) that is adapted to lacerate tissue such as the native valve leaflets 14 and/or artificial leaflet material that may be bovine pericardium, porcine pericardium, or even polymeric.

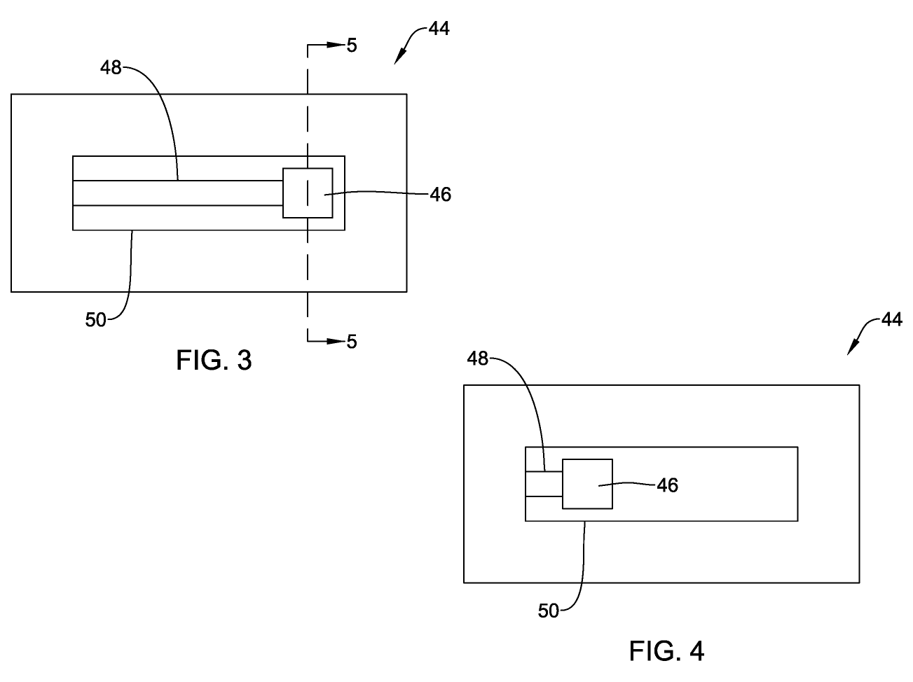

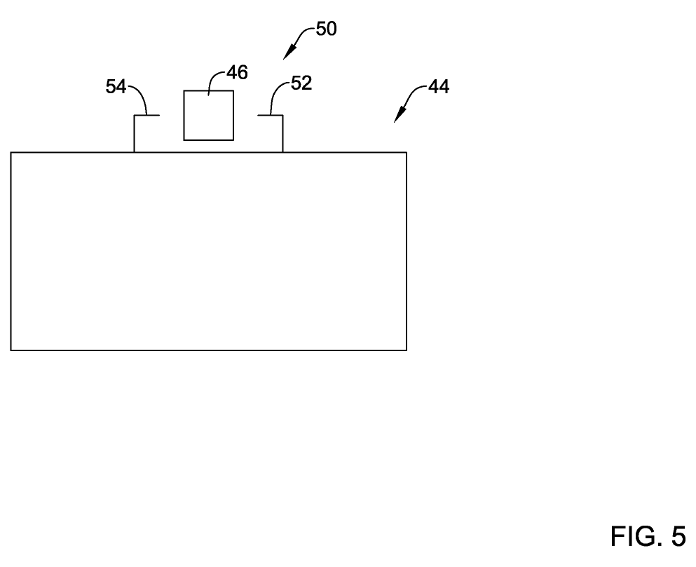

FIGS. 3 and 4 are schematic views of the expandable element 44, including a laceration member 46. FIG. 5 is a cross-sectional view of the expandable element 44, taken along the line 5-5 of FIG. 3. The laceration member 46, which may cut electrically or mechanically, is coupled with a translation member 48 that may be translated in order to cause the laceration member 46 to translate relative to the expandable element 44. In some cases, the expandable element 44 includes a guideway 50 in which the laceration member 46 is able to translate in response to the translation member being translated relative to the guideway 50. In some cases, the guideway 50 may be considered as defining a predetermined path along which the laceration member 46 is able to move. In some cases, the predetermined path may be considered as being a straight line. In some cases, the predetermined path may be a curved path, for example, or an undulating path. In some cases, the predetermined path may have a “U” or “V” shape, which may include having two distinct electrodes that puncture the valve leaflet 14 at the same spot, then drawn along respective paths. In some cases, the two electrodes could be actuated simultaneously or sequentially. Because the expandable element 44 secures the valve leaflet 14 in position, the two electrodes will have good tissue contact.

In some cases, the guideway 50 may be thought of as a track disposed on the expandable element 44. In some cases, as seen in FIG. 5, the guideway 50 may include a lumen 52 in which a slot 54 is formed to allow the laceration member 46 to extend out of the lumen 52 for lacerating tissue. In some cases, the guideway 50 may be polymeric or metallic, and may be secured in position relative to the expandable element 44 using a variety of techniques including thermal deposition, printing, adhesive bonding, casting, molding, and others. In some cases, the guideway 50 may include a frame that is rigid enough to define the predetermined path, and a thinner wider attachment portion that secures the guideway 50 to the expandable element 44. In some cases, the frame and attachment portion may be polymeric in order to prevent current leakage and maintain current density. The attachment portion would be thin and flexible in order to accommodate balloon collapse and folding.

In some cases, the translation member 48 may be a pull wire that can be pulled proximally in order to cause the laceration member 46 to translate proximally within the guideway 50. As an example, the translation member 48 may be pulled proximally in order to translate the laceration member 46 from its position shown in FIG. 3 to its position in FIG. 4. In some cases, the translation member 48 may be a push wire that can be pushed distally in order to cause the laceration member 46 to translate distally within the guideway 50. As an example, the translation member 48 may be pushed distally in order to translate the laceration member 46 from its position shown in FIG. 4 to its position in FIG. 3. In some cases, the laceration member 46 may be adapted to extend radially outwardly from the guideway 50 so that the laceration member 48 is able to contact and lacerate valve leaflet material. In some cases, the laceration member 46 may be adapted to extend above the guideway 50 a distance equal to or greater than a thickness of the tissue that will be lacerated. As an example, the laceration member 46 may be adapted to extend 0.020 inches (0.5 millimeters) to 0.079 inches (2 millimeters) above the guideway 50.

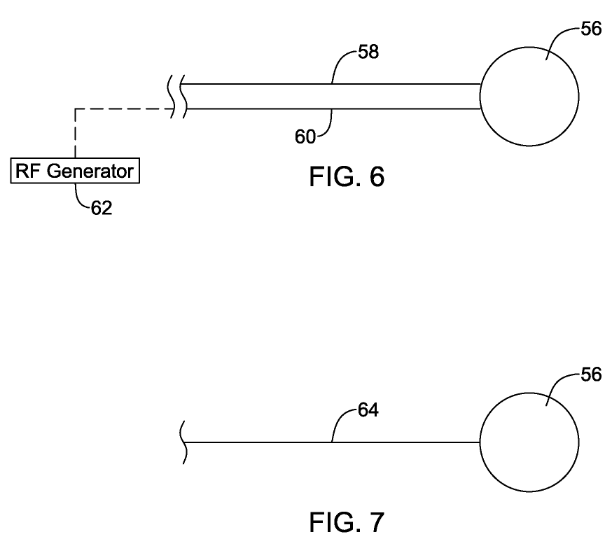

In some cases, the laceration member 46 may be adapted to cut tissue using RF (Radio Frequency) energy that is supplied to the laceration member 46. FIG. 6 is a schematic view of an RF laceration member 56 that is coupled to a translation member 58. In some cases, the RF laceration member 56 may be considered as being a point electrode. A point electrode can provide a greater current density relative to a larger or elongate electrode. In some cases, the RF laceration member 56 may be a rounded electrode. In some cases, the RF laceration member 56 may be considered as being an electrocautery electrode. The translation member 58 may be a pull wire or a push wire, for example. In some cases, the RF laceration member 56 is also coupled to an electrical conductor 60 that can provide RF energy to the RF laceration member 56. The electrical conductor 60 may extend to an RF generator 62. In some cases, the RF laceration member 56 may be coupled with a translation member 64 that also provides an electrical connection between the RF laceration member 56 and the RF generator 62 (FIG. 6), as shown in FIG. 7. While not shown, in some cases, a proximal portion of the translation member 64 may include a part of the translation member 64 that is coupled to a handle for translating the translation member 64 and may include a part of the translation member 64 that is coupled to the RF generator 62 (FIG. 6).

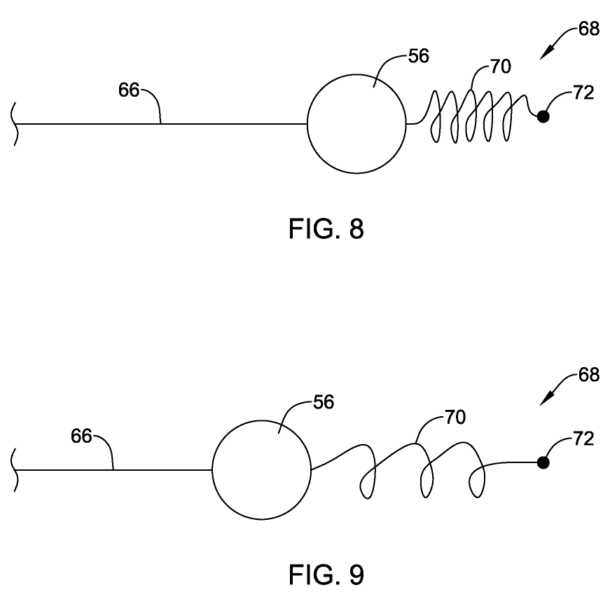

In some cases, a translation member may include a pull wire and a mechanism that causes the pull wire (and hence the laceration member) to return to a starting point. FIGS. 8 and 9 are schematic views of an illustrative translation member 66 that is coupled with the RF laceration member 56. A biasing mechanism 68 is also attached to the RF laceration member 56. In some cases, the biasing mechanism 68 may include a spring 70 that extends to an anchor point 72. The anchor point 72 may be embedded within part of the elongate shaft (such as the elongate shaft 40 shown in FIG. 2), for example. As shown in FIG. 8, the spring 70 is relaxed, and may be considered as being at an equilibrium condition. Pulling the translation member 66 (as shown in FIG. 9) results in the RF laceration member 56 moving proximally and stretching the spring 70 from its equilibrium configuration. In some cases, letting go of the translation member 66 may permit the spring 70 to pull the RF laceration member 56 from its position shown in FIG. 9 back to an equilibrium configuration as shown in FIG. 8. Accordingly, it is possible to move the RF laceration member 56 back and forth proximally and distally, as desired.

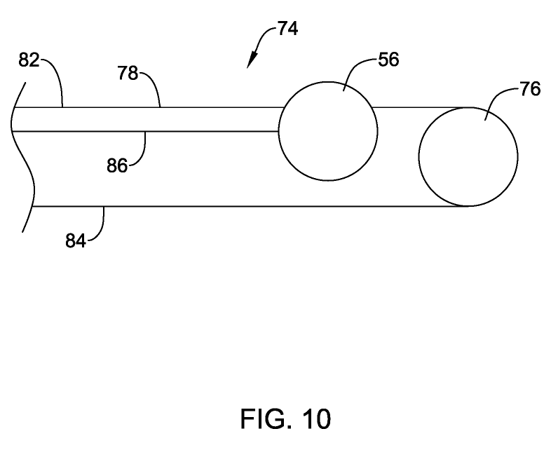

FIG. 10 is a schematic view of an illustrative mechanism 74 that includes a pulley 76. A translation member 78 is coupled with the RF laceration member 56 and wraps around the pulley 76. Accordingly, the RF laceration member 56 may be moved in a first direction by pulling on a first segment 82 of the translation member 78. The RF laceration member 56 may be moved in an opposing second direction by instead pulling on a second segment 84 of the translation member 78. The first segment 82 may be considered as part of the translation member 78 that is disposed on a first side of the pulley 76 while the second segment 84 may be considered as part of the translation member 78 that is disposed on a second side of the pulley 76. An electrical conductor 86 may be coupled to the RF laceration member 56 in order to provide RF energy to the RF laceration member 56. The mechanism 74 provides another way to be able to move the RF laceration member 56 distally and proximally as desired.

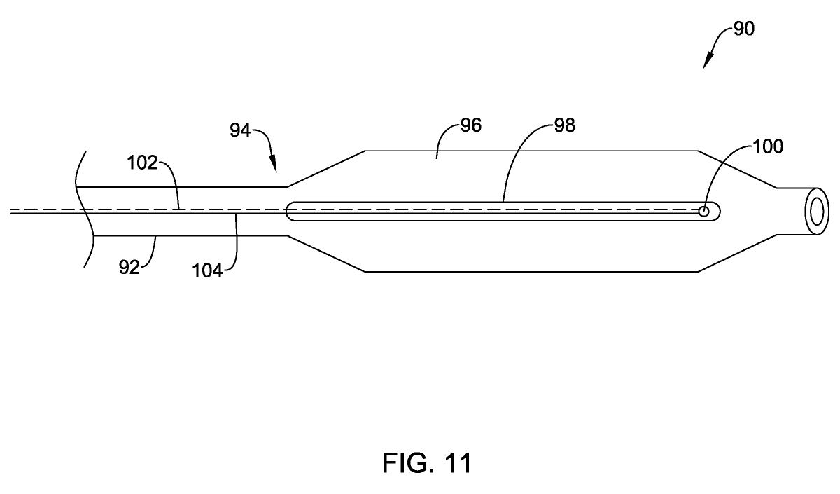

FIG. 11 is a schematic view of an illustrative medical device 90 that may be considered as being an example of the medical device 38. The medical device 90 includes an elongate shaft 92 extending proximally from a distal region 94 of the elongate shaft 92. An inflatable balloon 96 is coupled to the distal region 94 of the elongate shaft 92. While not shown, an inflation lumen extends through the elongate shaft 92 so that the inflatable balloon 96 may be deflated or inflated. As shown, the inflatable balloon 96 is shown in its expanded or inflated configuration. The medical device 90 includes a guideway 98 extending axially relative to the inflatable balloon 96. In some cases, a point electrode 100 is disposed relative to the guideway 98. A translation member 102 is coupled with the point electrode 100 and may be used to translate the point electrode 100 relative to the guideway 98. In some cases, as shown, an electrical conductor 104 may also be coupled with the point electrode 100 in order to provide RF energy to the point electrode 100. In some cases, while not shown, the translation member 102 may itself include an electrical conductor that provides RF energy to the point electrode 100 without requiring the separate electrical conductor 104. In some cases, when the inflatable balloon 96 is inflated, the inflatable balloon 96 pushes the point electrode 100 in a radially outward direction so that the point electrode 100 is better able to make contact with valve leaflet material.

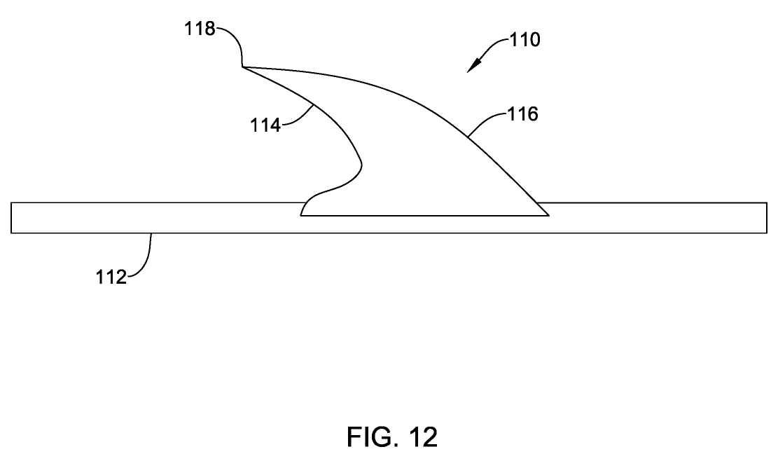

In some cases, the laceration member 46 (FIGS. 3 through 5) may be a mechanical element that is adapted to cut tissue. FIG. 12 is a schematic view of a mechanical laceration member 110 shown extending up through a guideway 112. The guideway 112 may be secured relative to an expandable element such as an inflatable balloon or an expandable framework such as a stent. The mechanical laceration member 110 may be coupled to a translation member or actuation mechanism (not shown) that allows the mechanical laceration member 110 to be translated. The aforementioned translation member may be a pull wire or a push wire, for example. In some cases, the translation member may be adapted to be able to move the mechanical laceration member 110 both distally and proximally.

The mechanical laceration member 110 includes several features. For example, the mechanical laceration member 110 includes a cutting blade 114 that is disposed along a proximal side of the mechanical laceration member 110. In some cases, the cutting blade 114 may be angled to form an acute angle with respect to a travel direction of the mechanical laceration member 110. In some cases, the cutting blade 114 may be curved. The mechanical laceration member 110 may include a rounded atraumatic surface 116 along a distal side of the mechanical laceration member 110. In some cases, a medical device including the mechanical laceration member 110 may be advanced distally to a desired treatment site such as the valve leaflets 14 (FIG. 1). Because of the rounded atraumatic surface 116, the mechanical laceration member 110 does not lacerate or otherwise cut tissue when moving distally. The cutting blade 114 cuts tissue when the mechanical laceration member 110 is moving proximally. In some cases, the mechanical laceration member 110 also includes a puncture feature 118 that may be used to puncture through the valve leaflet 14. Once the mechanical laceration member 110 has penetrated through the valve leaflet 14, the valve leaflet 14 may be lacerated by moving the mechanical laceration member proximally relative to the valve leaflet 14. In some cases, the mechanical laceration member 110 may remain underneath the guideway 112 until the underlying expandable element is expanded. In some cases, a removable sheath may help to hold the mechanical laceration member 110 underneath the guideway 112 until cutting is desired.

The materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel- copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result. In some cases, radiopaque markers may be incorporated into the medical devices described herein. In some cases, the electrocautery electrodes described herein may include radiopaque markers, or may themselves be formed of a radiopaque material. Positioning the electrocautery electrode translationally and rotationally can be important in being able to lacerate a valve leaflet centered on the coronary ostia behind the valve leaflet.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure’s scope is, of course, defined in the language in which the appended claims are expressed.

What is claimed is:

1. A medical device for lacerating valve leaflets, the medical device comprising:
an elongate shaft extending proximally from a distal region;
an expandable element secured to the distal region, the expandable element moveable between a collapsed configuration and an expanded configuration;
a guideway extending along the expandable element;
a laceration member slidingly disposed within the guideway; and
a translation member extending relative to the elongate shaft and coupled to the laceration member such that translating the translation member causes the laceration member to be translated relative to the guideway.

2. The medical device of claim 1, wherein the expandable element comprises an inflatable balloon.

3. The medical device of claim 1, wherein the laceration member comprises an electrocautery electrode.

4. The medical device of claim 1, wherein the laceration member is adapted to lacerate a valve leaflet using RF energy.

5. The medical device of claim 3, wherein the translation member is adapted to provide an electrical connection with the electrocautery electrode.

6. The medical device of claim 3, further comprising an electrical conductor that provides an electrical connection with the electrocautery electrode.

7. The medical device of claim 1, wherein the laceration member comprises a cutting blade.

8. The medical device of claim 7, wherein the cutting blade is disposed along a proximal region of the laceration member and a distal region of the laceration member is atraumatic.

9. The medical device of claim 7, wherein the laceration member further comprises a puncture feature.

10. The medical device of claim 1, further comprising a lumen accommodating the translation member therethrough, and the guideway comprises a slot formed along the lumen.

11. The medical device of claim 1, wherein the expandable element urges the laceration member into contact with the valve leaflet when the expandable element is in the expanded configuration.

12. The medical device of claim 1, wherein the valve leaflets comprise native valve leaflets.

13. A medical device for lacerating valve leaflets, the medical device comprising:
an elongate shaft extending proximally from a distal region;
an expandable element secured to the distal region, the expandable element moveable between a collapsed configuration and an expanded configuration;
an RF laceration member disposed relative to the expandable element; and
an actuation mechanism coupled to the RF laceration member to move the RF laceration member along a predetermined path relative to the expandable element.

14. The medical device of claim 13, further comprising a guideway defining the predetermined path.

15. The medical device of claim 13, wherein the RF laceration member comprises a round electrode.

16. The medical device of claim 13, wherein the actuation member comprises a pull wire.

17. The medical device of claim 13, wherein the actuation member comprises a push wire.

18. A medical device for lacerating valve leaflets, the medical device comprising:
an elongate shaft extending proximally from a distal region;
an expandable element secured to the distal region, the expandable element moveable between a collapsed configuration and an expanded configuration;
a mechanical laceration member disposed relative to the expandable element; and
an actuation mechanism coupled to the laceration member to move the mechanical laceration member along a predetermined path relative to the expandable element.

19. The medical device of claim 18, further comprising a guideway defining the predetermined path.

20. The medical device of claim 18, wherein the mechanical laceration member comprises:
an atraumatic edge along a distal region of the mechanical laceration member; and
a cutting blade along a proximal region of the mechanical laceration member.
